(19) United States
(12) Patent Application Publication
(10) Pub. No.: US 2015/0245800 A1
(43) Pub. Date: Sep. 3, 2015

(54) Method for Detection Of An Abnormal Sleep Pattern In A Person

(71) Applicant: Danmarks Tekniske Universitet, Kgs. Lyngby (DK)

(72) Inventors: Helge Bjarup Dissing Sørensen, Graested (DK); Julie Anja Engelhard, Copenhagen C (DK); Poul Jørgen Jennum, Farum (DK); Søren Rahn Christensen, Vallensaek Strand (DK); Lars Arvastson, Malmo (SE)

(21) Appl. No.: 14/422,833

(22) PCT Filed: Aug. 20, 2013
(86) PCT No.: PCT/EP2013/067297

§ 371 Date: Feb. 20, 2015

(30) Foreign Application Priority Data
Aug. 20, 2012 (EP) 12181048.5
Jun. 28, 2013 (EP) 13174352.8

(51) Int. Cl. A61B 5/00; A61B 3/113; A61B 5/0496

(52) U.S. Cl. CPC A61B 5/7282; A61B 5/0496; A61B 5/4812; A61B 5/4815; A61B 5/7264; A61B 3/113; A61B 5/4082

(57) ABSTRACT

The present disclosure relates to a method for detection of an abnormal sleep pattern based on a dataset of Electrooculography (EOG) signals obtained from a sleeping subject over a time interval, the method comprising the steps of dividing the time interval into a plurality of subintervals, each subinterval preferably corresponding to a sleep epoch, classifying each subinterval in terms of sleep stages, thereby obtaining a temporal sleep stage pattern, wherein a subject having an uncharacteristic temporal distribution of sleep stages is characterized as having an abnormal sleep pattern.

The present invention relates to a system and a method for detection of abnormal sleep pattern based on a dataset of Electrooculography (EOG) signals, and further to systems and methods for assisting in detecting neurodegenerative disorders such as Parkinson's.

### BACKGROUND OF INVENTION

Synucleinopathies are neurodegenerative disorders characterized by Lewy bodies and include Parkinson's disease, dementia with Lewy bodies and multiple system atrophy.

Parkinson's disease (PD) is a degenerative disorder of the central nervous system. The prevalence of PD is approximately 0.5% to 1% among people 65 to 69 years of age, rising to 1% to 3% among those aged 80 years or older. The neurodegeneration occurring in PD is irreversible and there is currently no cure for the disease.

The most obvious symptoms of PD are movement-related and include unilateral tremor, rigidity, akinesia and postural instability. Later, cognitive and behavioral problems may arise, with dementia commonly occurring in the advanced stages of the disease. Other symptoms include sensory, sleep and emotional problems.

Diagnosis of PD is currently based on the clinical manifestation of the motor symptoms, and treatments are directed at managing clinical symptoms. When the diagnosis is made based on the manifestation of the motor symptoms, the brain is already severely affected as the motor symptoms of PD arise from the loss of dopamine-generating neurons in the substantia nigra.

There are currently no reliable screening techniques available, which are capable of detecting PD in its very early stages, i.e. before motor symptoms appear. Such early screening techniques could potentially lead to the identification of more efficient treatments of Parkinson's disease and possible to a cure.

Rapid eye movement Sleep Behavior Disorder (RBD) is REM parasomnia characterized by REM sleep without atonia (RSWA) and/or dream enactment.

Therefore increased muscle tone and excessive phasic muscle-twitch activity of the submental or limb surface electromyography (EMG) may be measured.

RBD without any current sign of neurodegenerative disorder is designated as idiopathic RBD (iRBD). This term is questionable since RBD and other non-motor symptoms and findings are often observed in Parkinson's disease (PD) and atypical PD, such as multiple system atrophy (MSA) and Lewy Body Dementias (LBDs). A majority of patients suffering from synucleinopathies also experience sleep disturbances —, more than 50% of the subjects diagnosed with iRBD will develop a synucleinopathy within 5-10 years. Correct detection of RBD is therefore highly important, provided that neuroprotective treatment becomes available.

Rapid eye movement sleep behaviour disorder (RBD) affects about 0.4% of adults, 0.5% of older adults, 33% of patients with newly diagnosed Parkinson's disease, and 90% of patients with multiple system atrophy. Consequences can include injury to the patient, threats to the safety of a bed partner, and inability to share a bed with a partner. Diagnosis is important because the condition responds well to treatment, most often with clonazepam. Moreover, RBD may be a harbinger for neurodegenerative conditions such as Parkinson's disease (PD), multiple system atrophy (MSA), or dementia with Lewy bodies (DLB), which together comprise the alpha-synucleinopathies. In the absence of RBD, REM sleep without atonia may also signal increased risk for alpha-synucleinopathies.

REM behaviour Disorder, dream enacting behaviour and abnormal muscle activity during REM sleep, may be early markers for neurodegenerative diseases, such as Parkinson's disease and atypical PD. More than 50% of the subjects diagnosed with RBD will develop PD within a time span of 5-10 years.

Hence, an improved support system for allowing health care persons to provide a diagnosis to patients as early as possible would be advantageous, and in particular a more efficient and/or reliable method for this would be advantageous. But there are numerous problems with manual staging of eye movements: Lack of scoring standard for staging eye movements, the discrete state model is unrealistic and inconsistent manual annotation and high inter-rater variability is observed. And supervised methods for scoring the different states of eye movements are unrealistic. However, unsupervised methods can learn structures directly from the data.

Also, an improved support system for easily investigating effect or potential effect or response of a drug/medicine may be advantageous. Further, a system and method for evaluating or investigating effects of drug dosage and dosage regimes may be advantageous.

Method in relation to sleep analysis include a method such as described in Kempfner J et al: “Automatic REM sleep detection associated with idiopathic rem sleep Behavior Disorder”, Engineering in medicine and biology society, EMBC, 2011 Annual International Conference of the IEEE, IEEE, 30 Aug. 2011, pages 6063-6066, but is merely concerned with providing a method for distinguishing between stages when the patient is in REM and when the patient is not in REM. There is not presented further analysis of the data.

### SUMMARY OF INVENTION

There is a need for identification of novel biomarkers for synucleinopathies allowing for an earlier detection of these diseases. Such early detection could potentially lead to the development of novel and more efficient treatments and eventually to a cure.

A first aspect of the invention therefore relates to a method for detection of an abnormal sleep pattern based on a dataset of Electrooculography (EOG) signals obtained from a sleeping subject over a time interval, the method comprising the steps of:
a) dividing the time interval into a plurality of subintervals, each subinterval preferably corresponding to a sleep epoch,
b) classifying each subinterval in terms of sleep stages, thereby obtaining a temporal sleep stage pattern,
   wherein a subject having an uncharacteristic temporal distribution of sleep stages is characterized as having an abnormal sleep pattern.

A further embodiment of the invention relates to a system having the means for carrying out the herein described methods.

The activation and control of eye movements are a complex interaction between cortical brain regions and midbrain and basal brain structures. An abnormal sleep pattern, e.g. in the form of abnormal form/density/timely distribution of eye movements during sleep, may therefore be an indicator for synucleinopathy or early stages thereof. A further aspect of the invention therefore relates to a method for identifying a subject having an increased risk of developing a synucleinopathy comprising detecting an abnormal sleep pattern according to the herein disclosed method, wherein a subject having an abnormal sleep pattern has an increased risk of developing a synucleinopathy. The subject is preferably identified before clinical onset of the synucleinopathy. The synucleinopathy is preferably selected from Parkinson's disease, Multiple System Atrophy and Dementia with Lewy Bodies, thus, the synucleinopathy may be Parkinson's disease. The subject is preferably identified before manifestation of one or more motor symptoms selected from the group consisting of tremor, rigidity, akinesia and postural instability. Furthermore, the subject is preferably identified before substantial neuro-degeneration has occurred.

A further aspect of the present disclosure relates to a method for analysing data relating to sleep and/or wake patterns in a person. The method may comprise pre-recording a set of EOG (Electrooculography) signals in a time interval and providing the data, applying a filter to the set of physiological signals so as to reduce noise from the set of physiological signals, dividing the time interval into sub-time intervals, wherein the duration of each subinterval is determined based on a criteria for the EOG signal, for each sub-time interval determine features, and determining an over-all or sub-time based classification based on features from one or more of the sub-time intervals. The pre-recorded data may be obtained from a number of electrodes positioned on a person in e.g. a sleep facility while being monitored by health care personnel.

The herein disclosed methods are particularly, but not exclusively, advantageous as an aid for health care persons assessing patients either having a diagnosed neurodegenerative disease or in the process of being assessed as having a neurodegenerative disease. The methods are tools providing information that is not available from conventional analysis methods. The methods are envisioned to be used in conjunction with other tools selected by the health care person.

The method according to the first aspect provides an improved analysis solving at least the technical problem of identifying features not discovered by the conventional method. Further, by implementing the present methods as a computer program extracting features from the data set in an efficient manner is possible, thereby reducing processing time and eradicate the need for converting the recorded electrical signals to paper based hypnograms which are then analysed by humans.

### DESCRIPTION OF DRAWINGS

FIG. 1 is a schematic illustration of steps of a method.

FIG. 2 is a schematic illustration of the obtained posterior probability of belonging to the diseased class.

FIG. 3 is a schematic illustration of the proportions of the classes, the number of subjects, and the corresponding mixing coefficients of each component found by the clustering method described in an analysis of Ems.

FIG. 4 is a schematic illustration of the 3D feature space, where the posterior probability of the classes for each point is represented by colors defined by proportions of blue (highest probability of control class), green (highest probability of iRBD class) and red (highest probability of PD class).

FIG. 5 is a schematic illustration of exemplary electrode sites on a person.

FIG. 6 is a schematic illustration of a system.

FIG. 7 is a schematic overview of the methodology used in the study in example 1.

FIG. 8 is a topic mixture diagram and the corresponding manually scored hypnogram for a control subject, i.e. normal subject (example 2).

FIG. 9 is a topic mixture diagram and the corresponding manually scored hypnogram for an iRBD patient (example 2).

FIG. 10 is a topic mixture diagram and the corresponding manually scored hypnogram for a patient with Parkinson's disease (example 2).

FIG. 11 shows the classification obtained in the study described in example 2. The best NB classification result was based on two features: “Certainty” and “Stability”. The decision boundary is illustrated by the white control area and dark patient area, and the 30 test subjects are marked with blue (control subject), green (iRBD patient) or red (PD patient) filled circles.

FIG. 12 shows a hypothesized development of Parkinson's disease.

FIG. 13 shows the distribution of the three features in the study described in example 2.

The figures show one way of implementing the present invention and is not to be construed as being limiting to other possible embodiments falling within the scope of the attached claim set.

The invention can be implemented by means of hardware, software, firmware or any combination of these. The invention or some of the features thereof can also be implemented as software running on one or more data processors and/or digital signal processors.

### DETAILED DESCRIPTION OF THE INVENTION

The individual elements of an embodiment of the invention may be physically, functionally and logically implemented in any suitable way such as in a single unit, in a plurality of units or as part of separate functional units. The invention may be implemented in a single unit, or be both physically and functionally distributed between different units and processors.

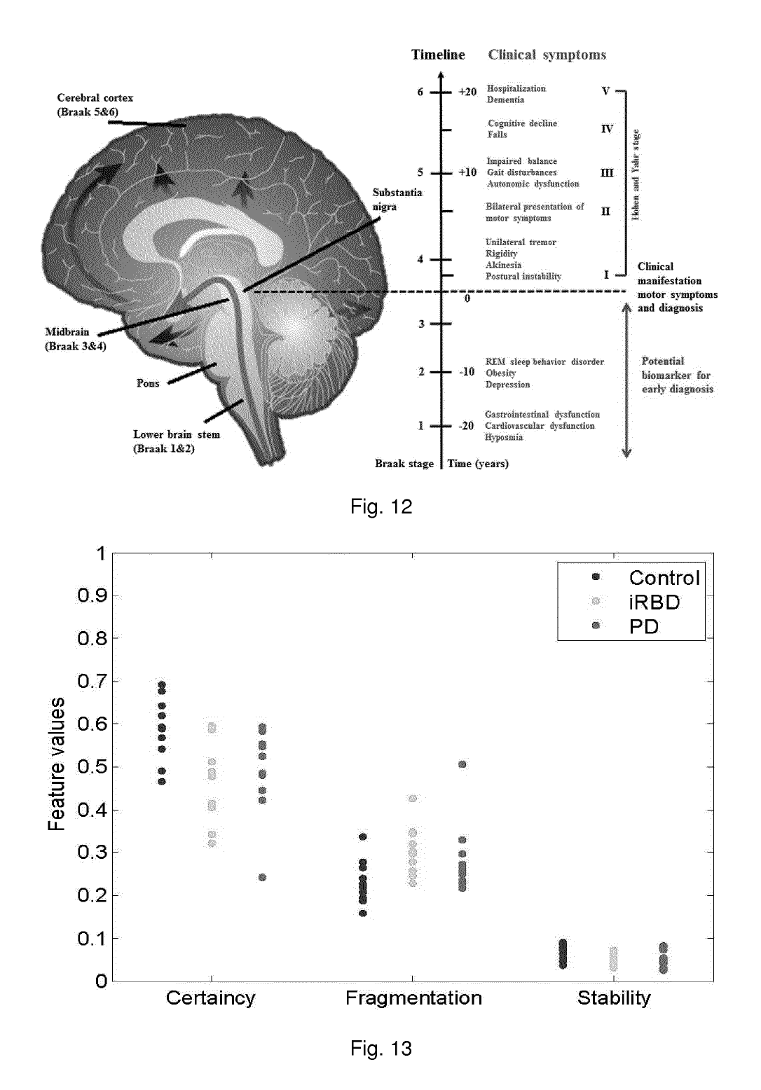

According to Braak et al., the evolution of Parkinson's Disease (PD) will involve the basal brain structures to start with (Braak stage I-II), and thereafter progress to the additional brain regions (Braak stage III-IV), cf. FIG. 12. During sleep, eye movements (EMs) are controlled by neurons located in the brain stem structures. No other studies have been focusing on analyzing EMs measured as electrooculography (EOG) during sleep, and for that reason, it was therefore hypothesized in a previous study (pilot study) that patients with iRBD and especially patients with PD will reflect abnormal form of EMs during sleep. In the pilot study, a subset of features holding the means and standard deviations across all sleep epochs of different decomposed Wavelet sub-bands was chosen based on a cross-validated Shrunken Centroids Regularized Discriminant Analysis (SCRDA). The classification of the subjects was done using the same method reaching a sensitivity of 95%, a specificity of 70% and an accuracy of 86.7%. The optimal subset of features was found to hold two features reflecting REMs and two features reflecting EMG activity, revealing that EMs hold potential of being a biomarker for PD. The purpose of the present disclosure is to see how good performance a classifier can obtain using only features reflecting EMs. Three of the high ranked features in the previous study reflecting slow EMs (SEM) and rapid EMs (REM) are analyzed looking at each sleep epoch rather than the mean and standard deviation across all sleep epochs.

In a study of the method according to the present invention the recording of EMs was done by EOG, which is based on a potential difference between the anterior (cornea) and the posterior (retina) point of the eyeball. In that way, the eye acts as a dipole in which the cornea is positive and the retina is negative. By placing electrodes besides each outer canthus, the EMs will be registered as positive potentials by the electrode nearest the cornea and as negative potentials by the electrode nearest the retina. Because of the simultaneous movement of the eyeballs, the EMs registered at the left and right EOG electrode will always appear synchronic and anti-correlated. The timely distribution of eye movements during sleep is advantageously approached and evaluated by categorising the eye movements into the following states: Slow Eye Movements (SEM), No Eye Movements (NEM) and Rapid Eye Movements (REM).

The method and system preferably analyses EOG-signals from an individual that has been under examination of a health care person. The method and system provides information that alleviates the examination of the patient.

A set of EOG-signals may be recorded by placing one or more electrodes near the eye region of a person to be monitored. The electrodes then record electrical signals originating from movement of the eye. The studies described herein indicate that by using EOG data (only) the specificity in categorizing eye movements, and thereby potentially providing early diagnosis of synucleinopathies, may be improved.

The time interval may be a sleep period where the person has been connected to a device recording EOG signals, while the person is in e.g. a sleep clinic or at home or other area or room where the person is to be monitored. The time interval may be a shorter interval of the period wherein the person has been asleep and may encompass periods where the person has been awake.

The sub time intervals (subintervals) may be time intervals of fixed length or may be time intervals of different length. The time intervals may e.g. be the length of a sleep epoch, i.e. 30 seconds.

The signals recorded may be subjected to a filtering so as to reduce artefacts. The filter may be the same filter applied to all channels or the filtering step may include filtering each signal in the set of physiological signals in a specific way.

Throughout the present description the signals are described as being ‘EOG’-signals, which is an acronym for electrooculography. The signals may encompass other measures of eye movement and physiological derivations hereof, such as auto-oculography (AOG).

Early detection of PD may thus be provided by assessing sleep patterns based on analyzing EOG signals only. Classifying the sleep pattern may thus be an efficient method to identify RBD, iRBD, RSWA, etc. In one embodiment of the invention the applied electrodes are the left and right EOG (EOGL, EOGR), preferably only these two electrodes. The two EOG channels are used for monitoring eye movements.

Usually RSWA and iRBD are detected using manually scored hypnograms where each sleep epoch (typically 30 seconds) is scored and/or characterized with a discrete value, e.g. REM, NREM, SEM, wake, etc. When obtaining a hypnogram the subject is provided with additional physiological monitoring means, e.g. EMG electrodes, e.g. on the legs because the legs typically move during REM sleep without atonia. However, the legs may move too much for subjects with RSWA possibly causing the electrodes to fall off. As demonstrated in example 2 herein abnormal sleep patterns may be detected by using EOG electrodes only. By relying on EOG electrodes only the measurement procedure becomes much more cost efficient and simpler, thus more patients can be diagnosed for the same costs.

In one embodiment of the invention a filter is applied to the set of EOG signals so as to reduce noise from the set of EOG signals.

In one embodiment of the invention the sleep stages are selected based on characteristic eye movements, preferably selected from the group of: slow eye movements (SEM), rapid eye movements (REM) and none eye movements (NEM). A set of probabilities may be calculated for each subinterval for each sleep stage, wherein each subinterval is classified by this set of probabilities.

In one embodiment of the invention each subinterval is divided into a plurality of time segments. A plurality of EOG features may then advantageously be calculated for each time segment. The duration of a time segment may be between 0.1 and 10 seconds, or more than 0.1, 0.2, 0.4, 0.5, 0.6, 0.7, 0.8, 1, 1.5 or more than 1.9 seconds, or less than 10, 8, 6, 4, 3, 2, or 1 second, preferably the duration of the time segments is 2 seconds. The duration of the subintervals may be between 1 and 120 seconds, or more than 5, 10, 15, 20, 25, or 30 seconds, or less than 120, 100, 90, 80, 70, 60, 50, 40, or less than 30 seconds, preferably the duration of the subintervals is 30 seconds corresponding to the duration of a standard sleep epoch. The subintervals are preferably non-overlapping. However, the time segments may be overlapping or non-overlapping.

In one embodiment of the invention the EOG features are selected from the group of:

at least one feature representing the spectral power of the left EOG signal,

at least one feature representing the spectral power of the right EOG signal, and

at least one feature representing the cross-correlation between the left and right EOG signals.

In a further embodiment of the present disclosure a topic model is applied to “learn” structures during short EOG patterns, i.e. the time segments. Automatically found topics will then be alternatives to the EM stages REM, SEM and NEM. The EOG features are preferably discretized into a plurality of discrete values or symbols, such as two, three, five, six, seven, preferably four values or symbols, based on a number of predefined boundaries. This discretization is provided for each time segment. A topic is then advantageously assigned to each sleep stage whereupon a topic model can be applied, a topic model such as the Latent Dirichlet Allocation (LDA) model. This is provided to compute the distribution of topics in each subinterval and/or to compute the individual topic probability in each subinterval and/or to compute the distribution of the individual topic probabilities in each subinterval. One or more classifier features may then be calculated for the time interval based on the distribution of topics and a classifier model, such as the Naive Bayes (NB) classifier, can then be applied to the one or more classifier features.

This distribution of topics may therefore correspond to the share of sleep stages which may be perceived as very indicative for which class (i.e. control/normal, pre-stage synucleinopathy or actual synucleinopathy, such as Parkinson's disease) a patient belongs to. It is thought that the more advanced the disease in a patient is, the more the eyes will fluctuate throughout the entire time interval. Thus, the SEM, REM and NEM share, in each subinterval or in the overall time interval, may indicate the disease classification.

In one embodiment of the invention said one or more classifier features are selected from the group of:
at least one certainty classifier feature representing the amount of subintervals with a dominating topic, such as a topic with a probability higher than a predefined threshold,
at least one fragmentation classifier feature representing the amount of state shifts between topics within a subinterval when the dominating topic defines the state of a subinterval, and
at least one stability classifier feature representing the number of subintervals kept in a certain state when the dominating topic defines the state, preferably the normalized mean number of subintervals kept in a certain state when the dominating topic defines the state.

Finally the EOG signal dataset may be classified as exhibiting normal sleep pattern or abnormal sleep pattern based on the results of the classifier model. Further classification may be provided to classify an abnormal sleep pattern into iRBD sleep pattern or synucleinopathy sleep pattern, such as Parkinson's sleep pattern. The herein discloses methods may advantageously be at least partly computer implemented thereby circumventing the lengthy procedure of manually scoring hypnograms. See example 2 wherein this classification is automated and used in a study of forty subjects.

In a further aspect the method may comprise for each sub-time interval determining a first, second and third value for a respective first, second and third class, and determining an over-all classification based on the first, second and third value for all sub-time intervals. In some embodiments only values for one or more of the sub-time intervals are calculated. The determination of an over-all classification may include combining the values rather than classifications for the sub-time intervals before determining the overall classification which establishes a more reliable classification. The method may further comprise discarding sub-time intervals where the patient is not in a sleep stage.

Advantageously the first, second and/or third values may represent probabilities for a class where the first class is ‘control’, the second class is a pre-Parkinson stage and the third class is Parkinson's disease. Probabilities are chosen as these measures are intuitively and easily combined. The stated classes are chosen as these encounter the whole scenario from normal/control to an advanced stage of the disease. Other terms may be used to describe the state of the persons.

Advantageously the method may further comprise applying a clustering method when determining each of the first, second and third values. The clustering method may advantageously be performed in feature space which is defined by features based on one or more sub-time intervals.

A clustering method can find and/or encompass areas of particular interest, often called components, in feature space and describe them by mathematical characteristics. Some areas/components will strongly relate to one of the three classes, and thereby be very indicative for that class or value.

In some instances it may be advantageous to apply a threshold level when determining each of the first, second and/or third values. Thresholding will control which inputs and/or mathematical characteristics should be used when determining the values.

Advantageously the thresholding may be performed on the components identified by the clustering method. Applying thresholding to the components is advantageous as it ensures that only the relevant amount/number of components is included, and thereby avoids the influence of components which will level out/smear out the differences between the components of interest.

The method may advantageously further comprise one or more steps of ranking the components identified by the clustering when determining each of the first, second and third values. Ranking the components will ensure that the most indicative components will have most influence. Preferably the ranking is performed on the components forming the basis of the values and not on the values themselves.

Advantageously the ranking may be based on the characteristics of the components identified by the clustering method. A characteristic could for instance be the prior probability of the three classes. The chosen characteristic indirectly defines what components are the most indicative ones.

Further the method may include recording and/or obtaining a set of (pre-recorded) physiological signals, the set of physiological signals being one or more of: muscle activity at or near the eye, eye movement morphology, muscle activity measured from one or more body parts including limbs and head, respiration frequency, heart rate, an electroencephalographycal (EEG) signal, an eletrocardiographycal (ECG) signal, and/or an electromyographycal (EMG) signal.

The set of signals may be used to derive further information regarding the EOG signal. The information may e.g. be used for filtering the EOG channel, or used in other ways to enhance the signals relating to the muscle movement near the eye.

Advantageously the features may include energy percentages in different frequency bands—and the common logarithm of the summed absolute signal values in different frequency bands. These features are advantageous as energy percentages are relative measures and the common logarithm of the summed absolute signal values are absolute measures. In this context the term ‘energy percentages’ may be construed as energy percentages of reconstructed signals holding different frequencies, where the percentages are of the total energy across the total/whole frequency content. By using different frequency bands it can be easier to distinguish between different physiological signals as well as between the classes.

Advantageously the features (or topics) may represent shares of one of three, or in some instances more than three, stages including: slow eye movements (SEM), rapid eye movements (REM) or none eye movements (NEM). The shares of these stages are perceived as very indicative for which class (i.e. control, pre-stage Parkinson's Disease or Parkinson's Disease) a patient belongs to. It is thought that the more advanced the disease in a patient is, the more the eyes will fluctuate throughout the entire time interval. Thus, the SEM, REM and NEM share, in each sub-time interval or in the overall time interval, will indirectly indicate the class.

Advantageously the method may comprise the features being determined using a data-driven topic model. A topic model is a statistical model revealing “topics” or “themes”, which describe the latent structure behind the generation of a collection of documents (see example 2). In an embodiment described later, a topic model is applied on data describing EMs during sleep, and each sleep epoch will be represented as a mixture of three different states for EMs. The three states are thought to be related to slow EMs (SEMs), rapid EMs (REMs) and no EMs (NEMs). By applying the topic model on three test groups of ten control subjects, ten iRBD patients and ten PD patients, it will be analysed how well the EMs from the patients fall into the normal states for EMs during sleep. By extracting three features from the topic models reflecting “certainty”, “fragmentation” and “stability”, the test subjects may be classified as “control” or “patient” by use of a Naive Bayes (NB) classifier.

Advantageously the stages may be defined by a correlation measure between two EOG signals simultaneously recorded at either side of the eyes. Two EOG electrodes placed on either side of the eyes, or at either side of one eye, will record the movement of the eyeballs as anti-correlated high amplitude signals and ECG- and EEG-artefacts as correlated, lower amplitude signals. EMG activity around the eyes will be recorded as uncorrelated, high-frequency signals. A correlation measure of these recordings will therefore capture the movement of the eyeballs in a robust way, and will be less sensitive to artefacts.

Advantageously the stages may be defined by certain frequency contents. The three stages SEM, REM and NEM may be seen as reflecting different frequencies and thereby state different frequency contents profiles.

Advantageously the frequency contents may be defined based on percentiles of the power values in different frequency bands of the signals. Defining the frequency contents by percentiles of the power values gives a more robust measure as it is less sensitive to outliers. The percentiles could e.g. be pairs defined by e.g. the 2^{nd} and 98^{th} percentile, the 5^{th} and the 95^{th} percentile or the 10^{th} and the 90^{th} percentile.

Advantageously the stages may be defined by specific amplitude levels. As an example the three stages SEM, REM and NEM may be seen as reflecting different amount of power/strength in the movement of the eyes/eyeballs and thereby reflect different amplitude profiles.

Advantageously the amplitude levels may be defined based on percentiles of the signal values. Defining the amplitudes by percentiles of the signal values gives a more robust measure compared to the “traditional” reading of the signal values as it is less sensitive to outliers originating from artefacts. The percentiles could be pairs defined by e.g. the 2^{nd} and 98^{th} percentile, the 5^{th} and the 95^{th} percentile or the 10^{th} and the 90^{th} percentile, depending on how much of the values are considered outlier.

This aspect of the invention is particularly, but not exclusively, advantageous in that the present invention may be accomplished by a computer program product enabling a computer system to carry out the operations of the apparatus/system of the first aspect of the invention when down- or uploaded into the computer system. Such a computer program product may be provided on any kind of computer readable medium, or through a network. The method according to the first aspect may thus be implemented in software so that it may be executed on a computer device. This may include a dedicated device incorporated in an apparatus having electrode input channels or a general purpose device having one or more interfaces for receiving inputs from electrodes, other sensor types or data representing electrode signals, e.g. recorded by a separate device.

A further aspect of the present invention relates to a system having one or more electrodes to be positioned on a subject or patient, the system further comprises a data collecting unit for recording data from the one or more electrodes, the system further comprises a data processing unit for processing the data recorded from the electrodes. The data processing unit is adapted or configured to carry out the steps of the herein described method. The data processing unit preferably comprises a software implementation allowing the data processing unit to perform the steps described in relation to the herein described method. The data processing unit may be adapted or configured to perform any select or all steps of the method, further the data processing unit may be adapted or configured to perform additional steps not described in the present specification.

The individual aspects of the present invention may each be combined with any of the other aspects. These and other aspects of the invention will be apparent from the following description with reference to the described embodiments.

In further embodiments the method may comprise a step of for each sub-time interval determining a first, second and third value for a respective first, second and third class, and determining an over-all classification based on the first, second and third value for all sub-time intervals.

The method may comprise having the first, second and third values representing probabilities for a class where the first class is ‘control’, the second class is a pre-Parkinson stage and the third class is Parkinson's disease.

The method may comprise applying a clustering method when determining each of the first, second and third values.

The method may comprise applying a threshold level when determining each of the first, second and third values.

The method may comprise performing the thresholding on the components identified by the clustering method, as described elsewhere in the present text.

The method may comprise ranking the components found by the clustering method when determining each of the first, second and third values.

The method may comprise performing the ranking based on the characteristics of the components identified by the clustering method.

The method may comprise recording a set of physiological signals, the set of physiological signals being one or more of: muscle activity at or near the eye, eye movement morphology, muscle activity measured from one or more body parts including limbs and head, respiration frequency, heart rate, an electroencephalographycal (EEG) signal, an eletrocardiographycal (ECG) signal, and/or an electromyographycal (EMG) signal.

The method may comprise using the features to represent energy percentages in different frequency bands—and the common logarithm of the summed absolute signal values in different frequency bands.

The method may comprise using the features to represent shares of one of three stages including: slow eye movements (SEM), rapid eye movements (REM) or none eye movements (NEM)

The method may comprise defining the stages by a correlation measure between two EOG signals simultaneously recorded at either side of the eyes. Alternatively or in combination herewith, the stages may be defined by a correlation measure between two EOG signals simultaneously recorded at either side of one eye.

The method may comprise the stages being defined by specific frequency contents, and/or the stages being defined by certain amplitude levels.

The method may comprise the amplitude levels being defined based on percentiles of the signal values.

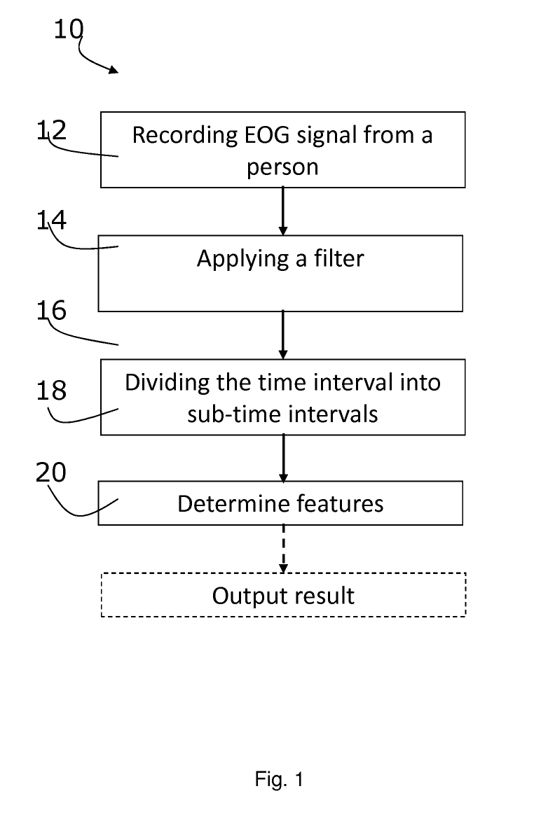

FIG. 1 schematically illustrates steps of a method 10. The method 10 is a method for assessing sleep and/or wake patterns in a person. The method 10 is at least useful for a health care person assessing whether a person have some degree of a neurodegenerative disease, in particular the method 10 is useful when assessing the person's likelihood or risk of Parkinson's Disease. The method 10 comprises the step of recording 12 a set of EOG signals in a time interval, or alternatively obtaining a set of EOG signals pre-recorded in a time interval. In the alternative the step may include recording a single EOG channel or signal. The time interval could e.g. be the time that the person is in a test facility in a health care institution such as a hospital or the like. The time interval may be a part of a longer recording, e.g. some hours of a night's sleep. The method 10 comprises the step of applying a filter 14 to the EOG signal or signals so as to reduce noise from the EOG signals. The method 10 comprises the step of dividing the time interval into sub-time intervals 16. This is done for more accurately determining certain properties relating to the signals recorded. The method 10 comprises the step of, for each sub-time interval, determine features 18. The features may range from a single feature to several features. The number of feature may for instance be 1 to 5 features, such as 3 or 4 features. In other embodiments a higher number of features may be used. A range of features will be discussed below. The method 10 comprises the step of determining an over-all or sub-time based classification based on features from one or more of the sub-time intervals. The method 10 provides an output related to an over-all or sub-time based classification aiding a health care person to assess the risk or likelihood that the person has a neurodegenerative disease. The method 10 is believed to allow a health care person to accurately assess the risk of neurodegenerative disease earlier than possible with currently available methods. The method 10 may conclude with the result of the method being output to the health care person or operator of a system performing the method. The dashed lines in the figure indicates an, in some embodiments, optional step.

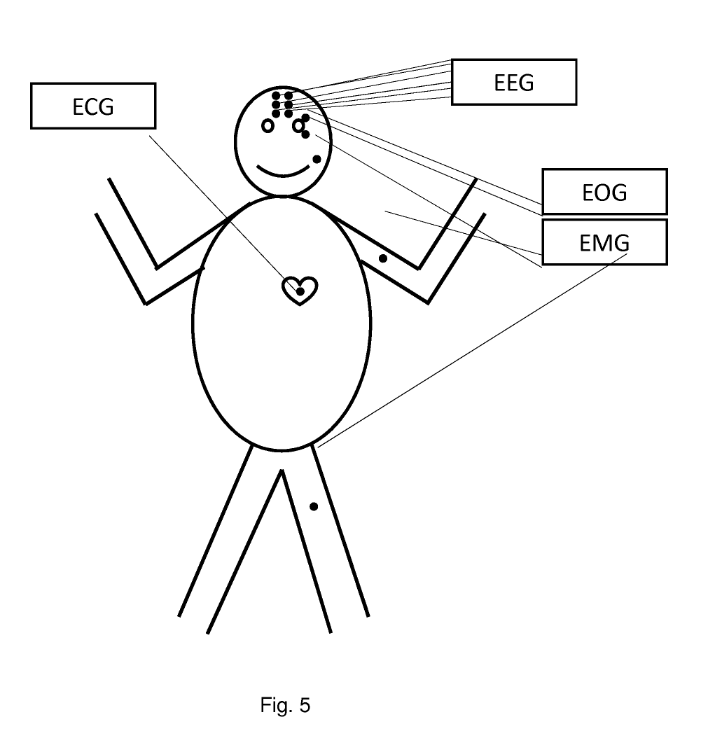

FIG. 5 is a schematic illustration of a person wearing a set of electrodes. The electrodes are placed as EEG, ECG, EOG and EMG electrodes. In certain embodiments not all three types of electrode positions are used. In particular embodiments not all electrodes of the EEG, EMG or ECG groups are used. In a presently preferred embodiment only data from the EOG is used as a basis for calculating.

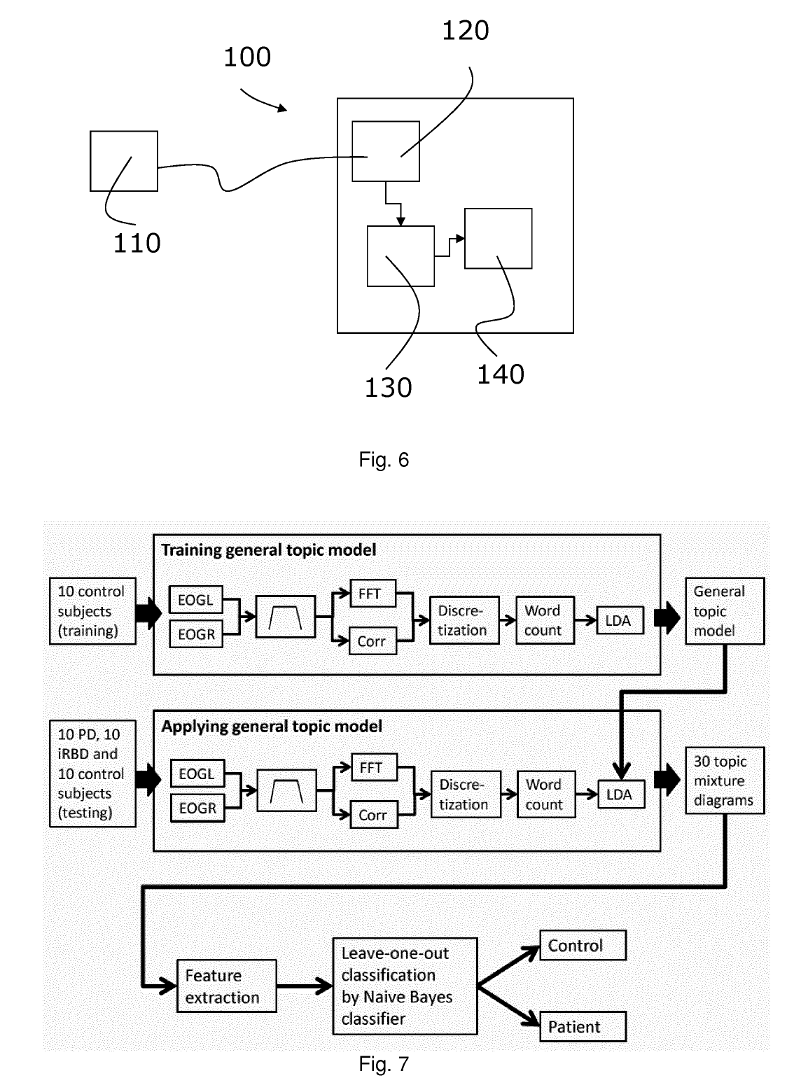

FIG. 6 is a schematic illustration of a system 100 having one or more electrodes 110 to be positioned on a subject or patient. The system 100 comprises a data collecting unit 120 for recording data from the one or more electrodes 110. The data collecting unit 120 may be an A/D converter. The A/D converter may comprise a filtering unit to perform pre- or post-conversion filtering to the one or more signals being recorded as described below.

The system 100 comprises a data processing unit 130 for processing the data recorded from the electrodes 110. In one embodiment one electrode is used, this, however, does not allow for use of correlation measurement as described elsewhere, other features and calculations are still possible using a single electrode. By use of two electrodes it may also be possible to measure or monitor a single eye of the person. The data processing unit 130 is adapted or configured to carry out the steps of the method as described above. The data processing unit 130 preferably comprises a software implementation of the method 10 allowing the data processing unit 130 to perform the steps described in relation to the method 10 above. The data processing unit may be adapted or configured to perform any select or all steps of the method 10, further the data processing unit 130 may be adapted or configured to perform additional steps not specifically described in the present specification. The system 100 comprises an output unit 140. The output unit 140 may be embodied as e.g. a screen, printer or other device providing a user with a visible output. The output unit 140 may alternatively be a transmitter for transmitting the output to another unit where the output is to be processed further or stored.

The system 100 may further comprise filters for filtering the signals, or simply signal, received from the electrodes 110. The filtering may be performed before the signals are A/D converted. The filtering may alternatively be performed after A/D conversion, or further alternatively both before and after NS conversion. The signals may, as mentioned elsewhere be combined with other signals, e.g. non-EOG signals for enhancing or extracting the EOG signal.

### Example 1

In a study patients enrolled were evaluated at the Danish Center for Sleep Medicine at Glostrup Hospital in Denmark. The evaluation of the patients included PSG, multiple sleep latency test and a comprehensive medical history and medication. Patients taking any anti-depressant drug, including hypnotics were excluded, though dopaminergic treatment was continued. Also, the quality of the PSG data was individually evaluated. If too much noise, such as disconnection, was present on the recordings making either the sleep stage scoring or the further analysis unreliable, the subject was excluded. A total of ten PD patients and ten iRBD patients were included in this study. Furthermore, ten age-matched control subjects without history of movement disorder, dream enacting behaviour or other former diagnosed sleep disorders were included as controls. Additionally, no medication known to affect sleep was acceptable. The demographic data for the two patient groups and the control group is seen in Table I.

TABLE I

| Patient | Total | Male/ | Age (μ ± σ) |
|---|---|---|---|
| groups | No. | Female | [years] |
| Controls | 10 | 5/5 | 59.8 ± 8.4 |
| iRBD | 10 | 8/2 | 59.0 ± 14.2 |
| PD + RBD | 10 | 6/4 | 63.2 ± 8.4 |

All controls underwent at least one night of PSG recorded outpatient, and all patients underwent at least one night of PSG recorded either outpatient or in-hospital. For the outpatient recordings, the PSG equipment was fitted at the clinic. The PSG recordings were performed in accordance with the sleep scoring standard stated in 2004 by the American Academy of Sleep Medicine (AASM) [C. Iber, “The AASM Manual for the scoring of Sleep and Associated Events”, American Academy of Sleep Medicine, 2007]. The EOG electrodes were placed one cm out and up (left) or down (right) from the outer canthus with reference to the mastoids. The sleep staging of all subjects were performed by experienced PSG technicians in accordance with the AASM standard staging every epoch of 30 seconds of PSG data into either REM sleep, three stages of non-REM sleep (N1, N2 or N3) or wake (W) resulting in a hypnogram of same length as the entire recording. The total number of scored epochs between lights off and lights on is seen in Table II below.

TABLE II

| Stage | Controls | iRBD | PD + RBD |
|---|---|---|---|
| Wake (%) | 1173 (12) | 1881 (18) | 1882 (19) |
| REM (%) | 2000 (21) | 1731 (16) | 1531 (15) |
| N1 (%) | 678 (7) | 1081 (10) | 1275 (13) |
| N2 (%) | 4443 (46) | 4881 (46) | 4073 (42) |
| N3 (%) | 1347 (14) | 1114 (10) | 1084 (11) |
| Sum (Σ %) | 9641 (100) | 10688 (100) | 9827 (100) |

The raw sleep data, hypnograms and sleep events were extracted from Nervus (V5.7, Cephalon DK, Nørresundby, Denmark) using the build-in export data tool. For further analysis, the data were imported to MATLAB (R2012a, The MathWorks, Natick, Mass., USA). The analysed data had a sampling frequency of 256 Hz.

In the article J. A. E. Christensen, R. Frandsen, J. Kempfner, L. Arvastson, S. R. Christensen, P. Jennum, and H. B. D. Sorensen, “Separation of Parkinson's patients in early and mature stages from control subjects using one EOG channel,” in Conference proceedings: Annual International Conference of the IEEE Engineering in Medicine and Biology Society. IEEE Engineering in Medicine and Biology Society. Conference, 2012, 28 features reflecting energies in different frequency bands were analyzed using the Discrete Wavelet Transform and a subset of these features was chosen based on the Shrunken Centroids Regularized Discriminant Analysis (SCRDA) method. It was shown that in the optimal subset of features, two reflect EMs and two reflect EMG activity. In this study, three of the 28 features reflecting EMs are analyzed further. In the article the features were calculated from the left side EOG signal. As recommended in the article, the features were in this study calculated based on a correlation signal EOGL-EOGR in order to reduce EEG artefacts. Also, the features were in this study analyzed in each sleep epoch, and not as the mean and standard deviation across all sleep epochs as they were in the article. Further description of the feature extraction as well as the SCRDA method can be found in the article.

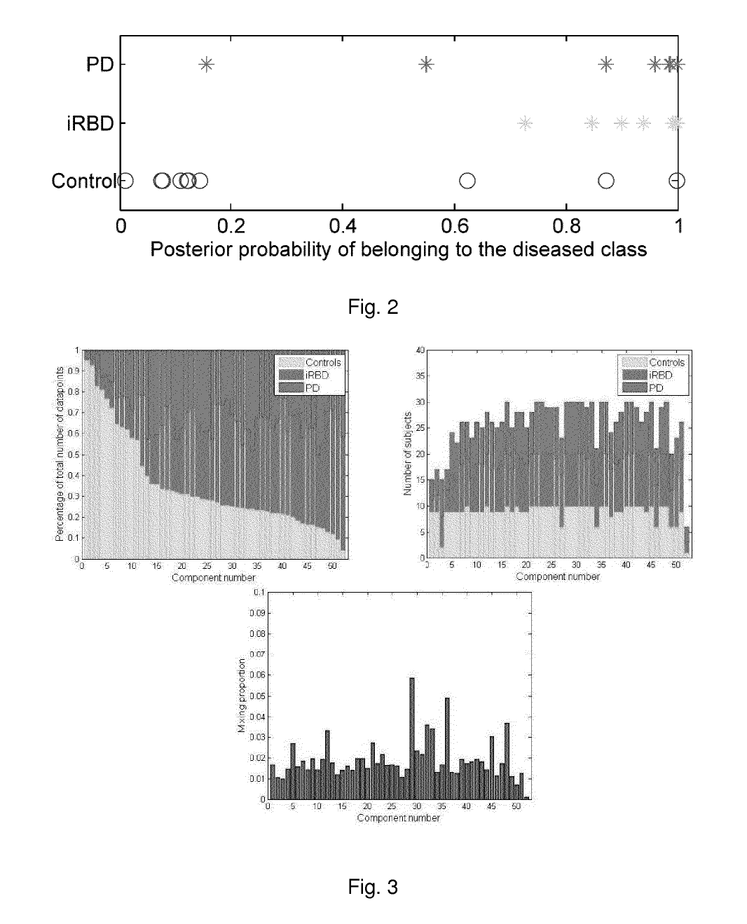

In FIG. 2 is seen the posterior probability of belonging to the diseased class. The posterior probabilities were calculated for each subject during the leave-one-out classification. The blue circles indicate the ten control subjects, the green stars indicate the ten iRBD patients and the red stars indicate the ten PD patients. When interpreting the result, it should be kept in mind that the iRBD and PD patients were treated as one class, i.e. the subset of features were found based on separation of controls and patients and not based on separation of controls, iRBD and PD patients. Following the criteria in the SCRDA method, it is seen from FIG. 2 that three control subjects are misclassified as diseased and one PD patient is misclassified as control, thereby yielding a sensitivity of 95%, a specificity of 70% and an accuracy of 86.7%.

In the further analysis of EMs during sleep, each sleep epoch from each of the 30 subjects were presented in a feature space defined by three features of the above mentioned. All three represent EMs, as one was derived as the energy percentage of the reconstructed detail subband d6, one as the energy percentage of the reconstructed detail subband d7 and one as the common logarithm of the summed absolute signal values of the reconstructed detail subband d8. The data was modeled by a Mixture of Gaussian (MoG) model, which was trained by a 10-fold cross validation technique. In this study, the estimation of the mean vector μₖ, the covariance matrix Σₖ and the corresponding mixing coefficient πₖ ε[0;1] was done by use of the Expectation Maximization algorithm for finding maximum likelihood solutions. The initial values for the means were set randomly to one of the samples in the dataset, and the Expectation Maximization algorithm was replicated 30 times, each time with a new set of initial parameters. The solution with the largest likelihood of the 30 replicates was chosen. The optimal number of components, K, was found by a 10-fold cross validation, where the data was split into 10 parts of equal size. For each of the ten runs, nine of these parts (training data) were used to train the model, and the negative log likelihood (N log L) was calculated for the held out part (test data). The mean and standard deviation of the N log L values for both the test data and training data across the ten runs were found for K=1, . . . , 60, and the minimum of the mean N log L of the test data was found at K=52. A final model with K=52 components was trained using all data and the proportions of the three classes (controls, iRBDs and PDs) across the 52 components were found on basis of the sleep epochs. It was also investigated how many subjects each of the components represented. The proportions of the classes, the number of subjects, and the corresponding mixing coefficients of each component is seen in FIG. 3.

The posterior probability of each class for each sleep epoch was determined by a leave-one-subject-out Naïve Bayesian approach, where the posterior probability p(c/x) of each of the held out sleep epochs from one subject was computed based on the conditional probability p(c/k) of the data from the 29 training subjects, where the prior probability for each component is given by the mixing coefficients p(k)=πₖ and the posterior probability of x given the component k is given by p(x/k)=N(x/μₖ,σₖ), which is the corresponding Gaussian density. In this way, a posterior probability of each of the classes for each sleep epoch was computed.

Classification of the subjects was done by assuming conditional independency (Naive Bayesian) between the posterior probabilities of the individual sleep epochs, and thereby combining the outputs systematically using the rules of probability. In this way, each sleep epoch was treated as a model with an independent output, and the subjects were classified by combining the models. Lastly, the class labeling was done simply by choosing the class with the highest posterior probability. In table III is seen the confusion matrix for classifying subjects into ‘control’ or ‘diseased’ using all components.

TABLE III

| | True | True |
|---|---|---|
| | diseased | control |
| Detected | 18 | 1 |
| diseased | | |
| Detected | 2 | 9 |
| control | | |

From table III is seen that only one control subject is misclassified as diseased and two patients are misclassified as controls, which yields a sensitivity, specificity and accuracy of 90%.

It was investigated how well the classification approach would perform if only including components with a posterior probability p(c/k) of any class above a given threshold by setting the mixing coefficients πₖ=0 for the components, that did not obey the given threshold for p(c/k) for any given class. The mixing coefficients for the components, that obeyed the threshold were normalized so Σπₖ=1. In table IV is seen the performance measures as well as the total number of included components for different thresholds.

TABLE IV

| | Total no. | | | |
|---|---|---|---|---|
| Threshold | in subset | Sensitivity | Specificity | Accuracy |
| None | 52 | 90% | 90% | 90% |
| p(c\|k) > 0.50 | 19 | 90% | 70% | 83.3% |
| p(c\|k) > 0.52 | 16 | 95% | 80% | 90% |
| p(c\|k) > 0.54 | 16 | 95% | 80% | 90% |
| p(c\|k) > 0.56 | 14 | 95% | 70% | 86.7% |
| p(c\|k) > 0.58 | 11 | 30% | 90% | 50% |
| p(c\|k) > 0.60 | 9 | 0% | 100% | 33.3% |

Other subsets of components were defined, where the components were ranked according to their posterior probability of each class. In table V is seen the performance measures for subsets of components, where the 1^{st}-5^{th} highest ranked component for each class is included in the MoG model.

TABLE V

| Component no. | | | |
|---|---|---|---|
| included in model | Sensitivity | Specificity | Accuracy |
| C: 1 | 95% | 90% | 93.3% |
| iRBD: 50 | | | |
| PD: 47 | | | |
| C: 1, 2 | 95% | 80% | 90% |
| iRBD: 50, 49 | | | |
| PD: 47, 34 | | | |
| C: 1, 2, 3 | 95% | 70% | 86.7% |
| iRBD: 50, 49, 51 | | | |
| PD: 47, 34, 32 | | | |
| C: 1, 2, 3, 4 | 95% | 70% | 86.7% |
| iRBD: 50, 49, 51, 40 | | | |
| PD: 47, 34, 32, 48 | | | |
| C: 1, 2, 3, 4, 5 | 90% | 80% | 86.7% |
| iRBD: 50, 49, 51, | | | |
| 40, 52 | | | |
| PD: 47, 34, 32, 48, 19 | | | |

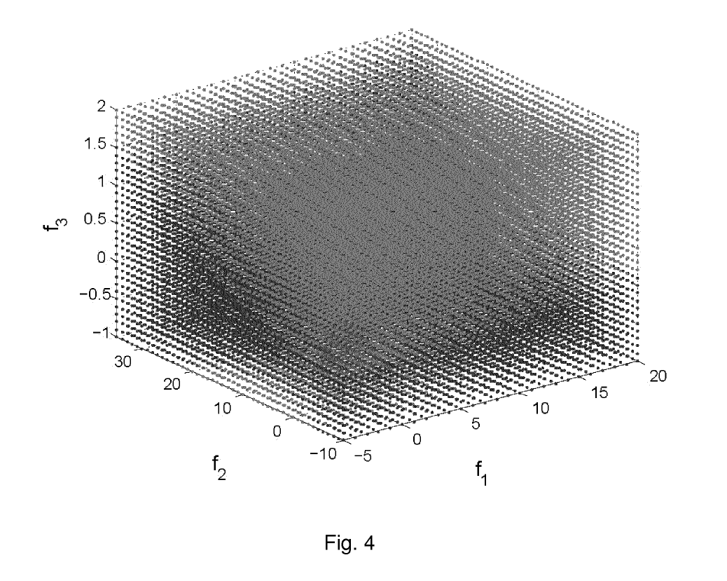

From table V is seen that a gain of 5% in sensitivity is achieved by using only three components compared to using all 52 components. The three components used are the ones that reflect the highest posterior probability of each class, being component number 1 (where p(control/k) is the major one), component 50 (where p(iRBD/k) is the major one) and component 47 (where p(PD/k) is the major one). In FIG. 4 is seen the 3D feature space, where the posterior probability p(c/x) for each point is calculated using the three components and represented by colors defined by proportions of blue (defined by p(control/x)), green (defined by p(iRBD/x)) and red (defined by p(PD/x)).

In the following one way of distinguishing between three classes will be discussed, and not only between not-diseased/healthy and partly/fully diseased. In table VI is seen a 3×3 confusion matrix computed from the classification results obtained using component number 1, 47 and 50.

TABLE VI

| | True | True | True |
|---|---|---|---|
| | control | iRBD | PD |
| Detected | 9 | 0 | 1 |
| control | | | |
| Detected | 1 | 10 | 8 |
| iRBD | | | |
| Detected | 0 | 0 | 1 |
| PD | | | |

The performance measures in the three class case (separating all three classes) yielded a mean sensitivity of 66.7%, a mean specificity of 83.3% and a mean accuracy of 77.8%. This cannot be considered a satisfactory result, but because the original aim of this study was to classify diseased from control subjects, it is not thoroughly investigated how to improve the three class case. One explanation of the poor result for the three class case can be the features used in this study, as they were found in a previous study, where the aim also was to classify patients from controls and not iRBD, PD and controls from each other. Another explanation of the poor result for the three class case can be found in the medical fields, as it could be that the neurons controlling EMs during sleep already are affected in very early stages of neurodegeneration, which is seen in iRBD patients. This could be why the EMs seen in PD and iRBD patients cannot be distinguished as both diseases are equally affected by the neurodegeneration in this area of the brain.

### Example 2

In a study forty subjects were enrolled. They were all evaluated at the Danish Center for Sleep Medicine at Glostrup Hospital in Denmark, and the evaluation of the patients included PSG, multiple sleep latency test and a comprehensive medical history and medication. The control subjects included have no history of movement disorder, dream enacting behaviour or other former diagnosed sleep disorders. The quality of the PSG data was individually evaluated, and recordings were excluded if the analysed channels were disconnected or continuously contaminated with artefacts. The demographic data for the groups is seen in the Table VII below.

TABLE VII

| Patient | Total | Male/ | Age (μ ± σ) |
|---|---|---|---|
| groups | No. | Female | [years] |
| Controls (for train) | 10 | 5/5 | 57.2 ± 8.1 |
| Controls (for test) | 10 | 5/5 | 59.8 ± 8.4 |
| iRBD (for test) | 10 | 8/2 | 59.0 ± 14.2 |
| PD (for test) | 10 | 6/4 | 63.2 ± 8.4 |

All subjects underwent at least one full night PSG according to AASM standards by use of different amplifier systems, where the lowest anti-aliasing filter cut-off frequency was 70 Hz. The EOG electrodes were placed one cm out and up (left) or down (right) from the outer canthus with reference to the right and left mastoid, respectively. The sampling frequency of the analysed sleep data was 256 Hz.

The overall methodology of this study is presented schematically in FIG. 7.

Ten control subjects selected to best match the patient groups in age were used to develop a general topic model. As input to the topic model, features extracted from band pass filtered EOG signals were given. By use of the general topic model, 30 topic mixture diagrams were obtained from ten control test subjects, ten iRBD test patients and ten PD test patients. Three features were extracted from these mixture diagrams, and by use of a standard NB classifier, the test subjects were classified as being either “patient” or “control”. Below follows a more detailed description of the steps seen in FIG. 7 illustrating a schematic overview of the methodology of this study.

Initially, both EOG signals were band pass filtered by a 4^{th} order Butterworth filter with cut-off frequencies (3 dB) at 0.3 Hz and 10 Hz. These cut-off frequencies were chosen to focus the topic model on EMs by suppressing the influence of the baseline drift, the EMG activity as well as some EEG activity measured at the EOG sites. Both EOG signals were divided into non-overlapping segments of length L, and for each of these segments, three features were computed, yielding a feature vector f(n) expressed as:

$$f ( n ) = [ \begin{matrix}{S}_{ll} ( n ) \\ {S}_{rr} ( n ) \\ {R}_{lr} ( n )\end{matrix} ]$$

where n denotes the segment index, Sₗₗ and Sᵣᵣ represents the spectral power computed by the fast Fourier Transform (FFT) below 5 Hz in the left and right EOG signal segment, respectively. Any EMs, whether it be SEMs, REMs or a combination of the two, are assumed to be in the range of 0-5 Hz. The Rₗᵣ represents the normalized cross-correlation coefficient between the left and right EOG signal segment given by:

$${R}_{lr} ( n ) = \frac{{σ}_{lr} ( n )}{\sqrt{{σ}_{ll} ( n ) {σ}_{rr} ( n )}}$$

where σₗₗ and σᵣᵣ denotes the variance of the left and right EOG signal segment, respectively, and σₗᵣ denotes the covariance of the left and right EOG signal segment.

As the EOG signals appear anti-correlated during EMs, it is assumed that Rₗᵣ will obtain negative values when REMs occur during REM sleep or wakefulness and when SEMs occur during N1 sleep. Background EOG should appear almost uncorrelated, and the high-amplitude EEG artefacts which can occur during deep sleep should appear correlated. The subject-specific median of the cross-correlation features was subtracted to align the values around zero.

The aim is to train a topic model by use of the Latent Dirichlet Allocation (LDA) model. To be able to use the features as input to such a topic model, the features were discretized on a per-subject basis. The spectral power features were given the values 1 to 4 based on boundaries set at each quartile for the full range of feature values for that specific subject. The cross-correlation features were discretized given values 1 to 4 based on boundaries set at [−0.7, 0, 0.7] for all subjects. These boundaries were set based on trial-and-error of best catching the EMs (at values below −0.7), and the EEG artefacts (values above 0.7) as well as the idea of having symmetric boundaries around zero.

The LDA method assumes that a “collection of documents” is derived from an underlying set of “topics”, and that the topics are defined as a set of related “words”. As the discretization in this study was done by symbols of 1 to 4, a word length of W is presented by either one of all combinations of W succeeding values of 1 to 4. The LDA assumes that each topic can be defined as a certain distribution over all of the available words. For each document in the collection of documents, a count is formed of the number of occurrences of each word, and as an end result a topic-by-document matrix X is found, describing the distribution over topics in each document.

The document length in this study was set to 30 seconds (comparable with a sleep epoch), yielding that each sleep epoch consisted of a total of 3*(30/L) instances. Different word lengths were tried (W=2, 3, 5), giving that the total number of available words was 3*(4̂W). The number of topics was set to T=3, in trying to reflect the different states (SEMs, REMs and NEMs) for EMs during sleep.

To train a general topic model, all the available sleep epochs in between lights off and lights on from ten control subjects were used as the collection of documents. By using data from control subjects only, a general “control topic model” was thereby trained. The topic model was applied on the three test groups (see Table VII), yielding a topic mixture diagram X holding the distribution of the three “control topics” in each sleep epoch from each of the subjects in the test data.

The aim of this study is to classify the 30 test subjects into either “control” or “patient” based on the topic mixture diagrams obtained when using a general topic model. For each test subject, three features were computed. The features reflect “certainty”, “fragmentation” and “stability”, and are defined as:

Feature 1—“Certainty”

The amount of epochs with a dominating topic of a probability higher than a given threshold. Normalization was done by dividing the number with the subject-specific total number of epochs. Feature 1 is expressed as,

$${f}_{1}^{p} = \frac{{\sum }_{k = 1}^{K} logical ( max ( {X}_{k}^{p} ) > th )}{K}$$

where K is the subject-specific total number of epochs and X^{p}ₖ is the EM topic mixture for epoch k in subject p. The threshold value th was defined as the one giving the highest mean Area Under Curve (AUC) when classifying the 30 test subjects using the leave-one-subject-out validation scheme.

Feature 2—“Fragmentation”

The amount of state shifts between topics when the dominating topic defines the state of an epoch. Normalization was done by dividing the number with the subject-specific total number of epochs. Feature 2 is expressed as,

$${f}_{2}^{p} = \frac{{\sum }_{k = 1}^{K - 1} logical ( max ( {X}_{k}^{p} ) \neq max ( {X}_{k + 1}^{p} ) )}{K}$$

Feature 3—“Stability”

The normalized mean number of epochs kept in a certain state when the dominating topic defines the state of an epoch. Feature 3 is expressed as,

$${f}_{3}^{p} = \frac{{\sum }_{m = 1}^{M} {e}_{m}^{new}}{M} with {e}^{new} = \frac{{e}^{old} - min ( {e}^{old} )}{max ( {e}^{old} ) - min ( {e}^{old} )}$$

where m is an index for a period, in where the epochs all have the same dominating topic, M is the subject-specific total number of such periods and e^{old} is a vector holding the M non-normalized numbers of epochs in each period.

As the topic mixture diagrams depend on the initialization of the LDA method, and as it was noticed that the feature values therefore slightly differed in between different runs on the same test subject, the three described features were computed for 20 different runs on the test data. The mean of the 20 feature values were used as the final feature values. Using the leave-one-subject-out approach, a standard NB classifier was used to classify the subjects into either “control” or “patient”. The classification was performed using all combinations of either one, two or all three feature values.

As mentioned earlier, different values were tried for the word length W (W=2, 3, 5) and for the segment length L (L=1, 3). The final topic model developed from the training dataset was chosen based on how well the NB classifier performed (according to accuracy) on the test dataset.

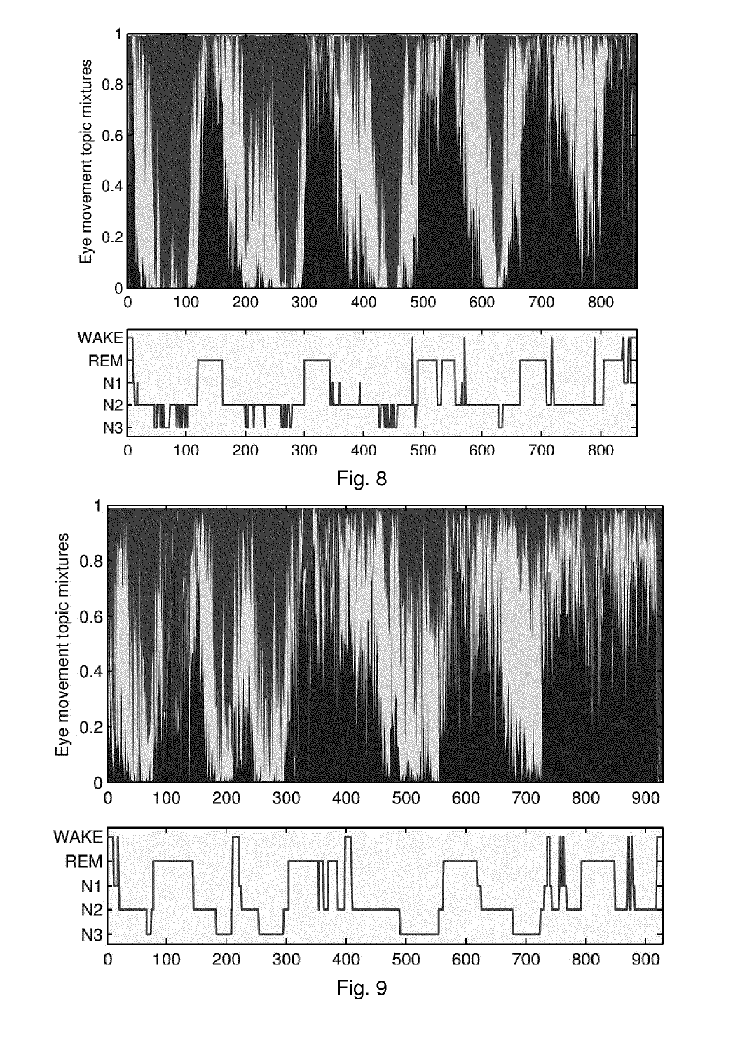

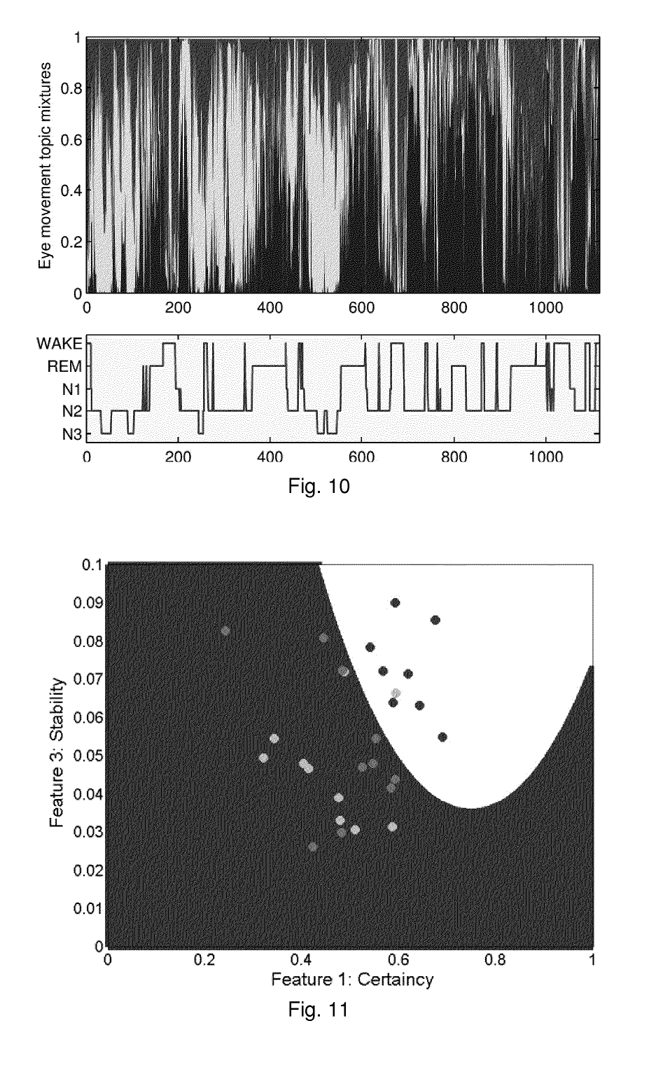

FIGS. 8, 9 and 10 present an example of topic mixture diagrams from a control subject, an iRBD patient and a PD patient, respectively. Each vertical coloured bin presents a sleep epoch, and the amount of each colour in a bin presents the individual topic probability. Remembering that the three topics are derived based on features reflecting EMs, it is seen, that the general topic model do recognize the characteristic temporal evolution of sleep. More specifically, the “blue” topic could be interpreted as having something to do with the REMs in REM sleep, whereas the “green” topic could be linked to SEMs and the “red” topic could be linked to NEMs. It is seen from the mixture topic diagrams in FIGS. 9 and 10, that not as many sleep epochs show a high certainty of either topic as compared to the control mixture diagram in FIG. 8. Interpreting the topics as just described, this observation lead to the conception that the EMs (both the REMs and SEMs) in the patients are less pronounced or less alike the EMs in control subjects. Other observations include the more abrupt transitions in between topics as well as the less structured and more fragmented profiles for the iRBD and PD patients compared to the control subjects. These observations are tried captured in the features “certainty”, “fragmentation” and “stability”.

A standard NB classifier was used to classify the subjects by the leave-one-subject-out validation approach, and it was found that the model, which obtained the highest mean accuracy, had a segment length of L=1 and a word length of W=3. This model used the two features “certainty” and “stability”, and in FIG. 11 the decision boundary is illustrated by the colours grey (classified as “patient area”) and white (classified as “control area”). The 30 test subjects are marked by red (PD patient), green (iRBD patient) or blue (control subject) filled circles. It is seen that two control subjects and one iRBD patient are misclassified, yielding a sensitivity of 95%, a specificity of 80% and an accuracy of 90%.

Training a general topic model based on sleep EOG from ten control subjects, revealed that the characteristic sleep cycles can be encompassed solely by use of features reflecting EMs. By applying the topic model on test data from ten other control subjects, ten iRBD patients and ten PD patients, a topic mixture diagram was obtained for each subject. Features reflecting “certainty”, “fragmentation” and “stability” of these diagrams were derived. The distribution of each of the three features for each patient group is shown in FIG. 13 with the control group (blue) to the left, iRBD patients (green) in the middle and Parkinson patients (red) to the right. There is a notable difference in the distribution of feature values between the three groups of subjects. It was found that by use of the two features “certainty” and “stability”, a simple NB classifier classified the subjects with a sensitivity of 95%, a specificity of 80% and an accuracy of 90% (FIG. 11).

The separability of the individual features as well as new features derived from the topic mixture diagrams should be further investigated. This study demonstrates with a data-driven, unsupervised approach that PD and iRBD patients reflect abnormal form and/or abnormal timely distribution of EMs during sleep. This study furthermore demonstrates that with a data-driven, unsupervised approach PD and iRBD patients reflect abnormal form and/or timely distribution of eye movements during sleep. This suggests involvement of brainstem nuclei in controlling eye movements.

### Further Details of the Invention

Although the present invention has been described in connection with the specified embodiments, it should not be construed as being in any way limited to the presented examples. The scope of the present invention is to be interpreted in the light of the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

The present invention may be characterised by the following points:
1. A method for assessing sleep and/or wake patterns in a person, the method comprising:
   recording a set of EOG signals in a time interval,
   applying a filter to the set of EOG signals so as to reduce noise from the set of EOG signals,
   dividing the time interval into sub-time intervals, for each sub-time interval determine features, and
   determining an over-all or sub-time based classification based on features from one or more of the sub-time intervals.
2. The method according to point 1, further comprising for each sub-time interval determining a first, second and third value for a respective first, second and third class, and determining an over-all classification based on the first, second and third value for all sub-time intervals.
3. The method according to point 2, wherein the first, second and third values represents probabilities for a class where the first class is ‘control’, the second class is a pre-Parkinson stage and the third class is Parkinson's disease.
4. The method according to point 2 or 3, further comprising applying a clustering method when determining each of the first, second and third values.
5. The method according to any one of the points 1-4, further comprising applying a threshold level when determining each of the first, second and third values.
6. The method according to point 5, wherein the thresholding is performed on the components identified by the clustering method.
7. The method according to point 4, further comprising ranking the components found by the clustering method when determining each of the first, second and third values.
8. The method according to point 7, wherein the ranking is based on the characteristics of the components identified by the clustering method.
9. The method according to any one of the points 1-8 wherein a set of physiological signals are recorded, the set of physiological signals being one or more of:
   muscle activity at or near the eye,
   eye movement morphology,
   muscle activity measured from one or more body parts including limbs and head,
   respiration frequency,
   heart rate,
   an electroencephalographycal (EEG) signal,
   an eletrocardiographycal (ECG) signal, and/or
   an electromyographycal (EMG) signal.
10. The method according to point 1, wherein the features represent energy percentages in different frequency bands—and the common logarithm of the summed absolute signal values in different frequency bands.
11. The method according to point 1, wherein the features represent shares of one of three stages including: slow eye movements (SEM), rapid eye movements (REM) or none eye movements (NEM)
12. The method according to point 11, wherein the stages are defined by a correlation measure between two EOG signals simultaneously recorded at either side of the eyes.
13. The method according to point 11, wherein the stages are defined by specific frequency contents.
14. The method according to point 11, wherein the stages are defined by certain amplitude levels.
15. The method according to point 14, wherein the amplitude levels are defined based on percentiles of the signal values.

1. A method for detection of an abnormal sleep pattern based on a dataset of Electrooculography (EOG) signals obtained from a sleeping subject over a time interval, the method comprising the steps of:
a) dividing the time interval into a plurality of subintervals, each subinterval preferably corresponding to a sleep epoch, and
b) classifying each subinterval in terms of sleep stages, thereby obtaining a temporal sleep stage pattern,
wherein a subject having an uncharacteristic temporal distribution of sleep stages is characterized as having an abnormal sleep pattern.

2. The method according to claim 1, wherein the sleep stages are selected based on characteristic eye movements, preferably selected from the group of: slow eye movements (SEM), rapid eye movements (REM) and none eye movements (NEM).

3. The method according to claim 1, further comprising the step of calculating a set of probabilities for each subinterval for each sleep stage, and wherein each subinterval is classified by this set of probabilities.

4. The method according to claim 1, further comprising the step of dividing each subinterval into a plurality of time segments.

5. The method according to claim 4, further comprising the step of calculating a plurality of EOG features for each time segment.

6. The method according to claim 5, wherein the EOG features are selected from
at least one feature representing the spectral power of the left EOG signal,
at least one feature representing the spectral power of the right EOG signal, or
at least one feature representing the cross-correlation between the left and right EOG signals.

7. The method according to claim 6,
further comprising the step of for each time segment discretizing said EOG features into a plurality of discrete values or symbols, such as two, three, five, six, seven, preferably four values or symbols, based on a number of predefined boundaries.

8. The method according to claim 1, further comprising the step of assigning a topic to each sleep stage and applying a topic model, such as the Latent Dirichlet Allocation (LDA) model, to compute the distribution of topics in each subinterval and/or to compute the individual topic probability in each subinterval and/or to compute the distribution of the individual topic probabilities in each subinterval.

9. The method according to claim 8, further comprising the step of calculating one or more classifier features for the time interval based on the distribution of topics and applying a classifier model, such as the Naive Bayes (NB) classifier, to said one or more classifier features.

10. The method according to claim 9, wherein said one or more classifier features are selected from:
at least one certainty classifier feature representing the amount of subintervals with a dominating topic, such as a topic with a probability higher than a predefined threshold,
at least one fragmentation classifier feature representing the amount of state shifts between topics within a subinterval when the dominating topic defines the state of a subinterval, or
at least one stability classifier feature representing the number of subintervals kept in a certain state when the dominating topic defines the state.

11. The method according to claim 10, further comprising the step of classifying the EOG signal dataset as exhibiting normal sleep pattern or abnormal sleep pattern based on the results of the classifier model.

12. The method according to claim 1, further comprising the step of classifying an abnormal sleep pattern into iRBD sleep pattern or synucleinopathy sleep pattern, such as Parkinson's sleep pattern.

13. The method according to claim 4, further comprising the step of applying a filter to the set of EOG signals so as to reduce noise from the set of EOG signals.

14. The method according to claim 13, wherein the duration of a time segment is between 0.1 and 10 seconds, or more than 0.1, 0.2, 0.4, 0.5, 0.6, 0.7, 0.8, 1, 1.5 or more than 1.9 seconds, or less than 10, 8, 6, 4, 3, 2, or 1 second, preferably the duration of the time segments is 2 seconds.

15. The method according to claim 1, wherein the duration of the subintervals is between 1 and 120 seconds, or more than 5, 10, 15, 20, 25, or 30 seconds, or less than 120, 100, 90, 80, 70, 60, 50, 40, or less than 30 seconds, preferably the duration of the subintervals is 30 seconds.

16. The method according to claim 4, wherein the subintervals are non-overlapping.

17. The method according to claim 16, wherein the time segments are overlapping or non-overlapping.

18. The method according to claim 1, wherein the method is based on analysis of EOG signals only.

19. The method according to claim 1, wherein the method is computer implemented.

20. A method for identifying a subject having an increased risk of developing a synucleinopathy comprising detecting an abnormal sleep pattern according to the method of claim 1, wherein a subject having an abnormal sleep pattern has an increased risk of developing a synucleinopathy.

21. The method according to claim 20, wherein the subject is identified before clinical onset of the synucleinopathy.

22. The method according to claim 20, wherein the synucleinopathy is selected from Parkinson's disease, Multiple System Atrophy or Dementia with Lewy Bodies.

23. The method according to claim 20, wherein the synucleinopathy is Parkinson's disease.

24. The method according to claim 23, wherein the subject is identified before manifestation of one or more motor symptoms selected from the group consisting of tremor, rigidity, akinesia and postural instability.

25. The method according to claim 20, wherein the subject is identified before substantial neurodegeneration has occurred.
